(19) United States
(12) Patent Application Publication
(10) Pub. No.: US 2004/0265267 A1
(43) Pub. Date: Dec. 30, 2004

(54) Crosslinked three-dimensional polymer network, method for preparing same, support material comprising same and uses thereof

(76) Inventor: Raphael Duval, Deauville (FR)

Correspondence Address: DAVID J. SERBIN, LAW OFFICE OF DAVID J. SERBIN, 1217 KING STREET, ALEXANDRIA, VA, 22314, US

(21) Appl. No.: 10/810,855

(22) Filed: Mar. 29, 2004

(30) Foreign Application Priority Data
Mar. 27, 2003 (FR) 03 03770

(51) Int. Cl. A61K 31/74

(52) U.S. Cl. 424/78.27

(57) ABSTRACT

The present invention relates to a crosslinked optically active three-dimensional polymer network consisting of at least one homochiral unit of at least one first selector and of at least one homochiral unit of at least one second selector of structure different from the first selector and optionally of at least one homochiral unit of at least one third selector of structure different from the first and from the second selector, the homochiral unit(s) of the first selector containing one polymerizable functional group, the homochiral unit(s) of the third selector containing at least one polymerizable functional group and the homochiral unit(s) of the second selector containing at least two polymerizable or crosslinkable functional groups, the homochiral units being chemically linked to one another, to a method for preparing thereof and methods using thereof.

The present invention relates to crosslinked three-dimensional polymer networks, to the method for preparing them, and also to optically active support materials containing said three-dimensional polymer networks.

The invention also relates to the use of these crosslinked three-dimensional polymer networks and also to the optically active supports for optically enriching chiral molecules, and more particularly for separating enantiomers by liquid, supercritical, gas or gas-liquid chromatography.

When they are used in a chromatographic process, the supports of the invention constitute homochiral stationary phases, or “CSPs”, and the technique used is then called chiral or enantioselective chromatography.

Chiral or enantioselective chromatography has experienced a considerable expansion over the last twenty years, both for applications in terms of analysis, but also for the industrial preparation of homochiral pharmaceutical molecules.

In fact, since the thalidomide tragedy in the 1960s, the health authorities of industrialized countries have gradually imposed regulatory restraints on industrial companies in the field of pharmacy, which must now support their dossier for a marketing authorization for new medicinal products with compared pharmacological and toxicological data for each homochiral or enantiomer molecule present in the future medicinal product.

Among the various homochiral stationary phases, or CPSs, which have been the subject of industrial developments, in order to produce homochiral molecules by preparative chromatographic resolution, polymeric selectors based on cellulose homopolymer derivatives (EP 0 147 804) or based on polymers having an asymmetric carbon atom in the principal chain (EP 0 155 637 B2) have until now constituted the most widely used technology.

Other selectors have also been the subject of considerable developments on an industrial scale, such as optically active polymers crosslinked in a network and chemically attached to a support (PCT/SE 93/01050) or also crosslinked but not necessarily chemically attached to a support (FR 98/11376, FR 98/11377, U.S. Pat. No. 6,042,723, EP 0 899 272 A1, EP 0 864 586 A2, WO 96/27615, WO 97/04011).

Other selectors have also been described, in particular in U.S. Pat. No. 6,277,782 and patent applications EP 985 682 and EP 656 331. These selectors consist of a single type of homochiral units which are monomers or polymers crosslinked by means of a nonchiral crosslinking agent or of a chiral, but not optically active, crosslinking agent as described in U.S. Pat. No. 6,011,149.

A hydrogel of chitosan and 2,3-dialdehydo-β-cyclodextrin has also been described in Chemical Reviews, 1998, Vol. 98, No. 5, page 1780.

However, there exists a real need for new optically active supports capable of allowing the separation of molecules exhibiting diverse chemical structures and exhibiting abilities for enrichment and for separation of enantiomers which are greater than those known and described until now, this ability being measured by the chromatography selectivity factor α.

After long and thorough research studies, the applicant company has found that these aims are achieved by using a crosslinked optically active three-dimensional polymer network according to the invention. The Applicant described chiral selectors which are formed by a specific cross-linked three-dimensional polymer network, in the patent application FR0112208.

The Applicant has further completed his searches and found numerous chiral selectors formed by a cross-linked three-dimensional polymer network.

The invention therefore relates, according to a first advantageous embodiment, to a crosslinked optically active three-dimensional polymer network consisting of one homochiral unit of at least one first selector and of at least one homochiral unit of at least one second selector of a structure different from the first selector,

the homochiral unitof the first selector containing one polymerizable functional group and the homochiral unit(s) of the second selector containing at least two polymerizable or crosslinkable functional groups,

the homochiral units being chemically linked to one another,

with the exclusion of the crosslinked three-dimensional polymer networks obtained by polymerization of (S)-glycidylmethacrylate and simultaneous cross-linking with (S,S)-2,3-butanediol dimethacrylate, or by polymerization of 3-([2-(S)-hydroxy]-N-benzylamino)propyl methacrylate and simultaneous cross-linking with (S,S)-2,3-butanediol dimethacrylate.

A “homochiral unit” represents a monomeric, oligomeric or polymeric compound which is homochiral.

The polymerizable or crosslinkable functional groups are in particular primary, secondary or tertiary hydroxyl groups, primary or secondary amine groups, sulfhydryl groups, ethylenic double bonds or aldehyde groups.

In the present application, the expression “homochiral units being linked to one another” is intended to mean the fact that the various homochiral units are linked to one another via bonds resulting from polymerization (homopolymerization or copolymerization) or from crosslinking. The polymerization is carried out by virtue of functional groups present on the homochiral units. The crosslinking, which allows the formation of a three-dimensional network, is carried out by virtue of said functional groups or, optionally, using a nonchiral crosslinking agent containing at least two polymerizable or crosslinkable functional groups. With polymerization, a linear chain is obtained, whereas with crosslinking, a three-dimensional assembly is obtained.

The oligomers or polymers are of natural origin (polysaccharides, proteins, DNA, etc.) or are obtained by homopolymerization of the same homochiral monomer. They may also be obtained by copolymerization of two homochiral monomers of different chemical structure. Optically active heteropolymers are then obtained.

The optically active heteropolymers or homopolymers consist of at least 11 homochiral units (Nomenclature et Terminologie en Chimie Organique [Nomenclature and Terminology in Organic Chemistry], September 1996, Techniques de l'Ingénieur [Techniques for the Engineer], 249, rue de Crimèe, 75019 Paris) and their related oligomers consist of 1 to 10 homochiral units which are identical for the homopolymers and homooligomers and different for the heteropolymers and heterooligomers.

By way of example, a β-cyclodextrin or cyclomaltoheptaose is a cyclic oligosaccharide (Chemical Reviews, 1998, Vol. 98, No. 5, p 1745) and therefore a homooligomer.

It is a chiral selector which is greatly used in the synthesis of chiral stationary phases for chromatography. It may be mono- and polyfunctional given that the cyclodextrin molecule contains 21 primary and secondary alcohol functions. As such, β-cyclodextrin has a perfectly defined optical rotation and is optically active.

In accordance with the invention, the homochiral units containing one polymerizable functional group are chosen from the group comprising in particular mono-6-O-(4-allyloxyphenylcarbamate)-hexakis-6-O-(3,5-dimethylphenylcarbamate)-di-heptakis-2,3-O-(3,5-dimethylphenylcarbamate)-β-cyclodextrin, 2-propynyl-tetra-O-acetyl-β-glucopyranoside, allyl-α-D-galactopyranoside, 1-O-allyl-2-deoxy-4,6-O-isopropylidene-2-(trifluoroacetamido)-α-D-galacto pyranoside, 7-allyl-7,8-dihydro-8-oxoguanosine, (R)-(+)-α-acryloxy-β,β-dimethylbutyrolactone, acrylamido-(L)-alanine ethyl ester, (2S,5R)-(+)-5-vinyl-2-quinuclidinemethanol, (2R,5R)-( )-5-vinyl-2-quinuclidinemethanol, quinine and quinidine.

In accordance with the invention, the homochiral units containing two polymerizable or cross-linkable functional groups are chosen from the group comprising in particular (R,R)-dithiothreitol (DTT), tartaric acid or derivatives thereof, such as N,N′-diallyltartramide (DAT), di-tert-butylbenzoyldiallyltartramide (DBBDAT), diacetyldiallyltartramide (DADAT), bi-derivatives of cyclodextrin, in particular β-cyclodextrin, such as bis-6A, 6D-O-(4-allyloxyphenylcarbamate)pentakis-6-O-(3,5-dimethylphenylcarbamate)di-heptakis-2,3-(3,5-dimethylphenylcarbamate)-β-cyclodextrin.

In accordance with the invention, the homochiral units containing more than two polymerizable or cross-linkable functional groups are chosen from the group comprising in particular three- and poly-dericvatives of cyclodextrin, in particular β-cyclodextrin, such as tetrakis-60-(4-allyloxyphenylcarbamate)tris-6-O-(3,5-dimethylphenylcarbamate)-heptakis-2,3-O-di-(3,5-dimethylphenylcarbamate)-β-cyclodextrin (T(AOPC-DMPC)), cellulose or derivatives thereof such as cellulose [6-(4-allyloxyphenyl)urethane, tris-2,3,6-[3,5-dimethylphenyl)-urethane (L(AOPC-DMPC)), chitosan or derivatives thereof.

The structural formulae of some of these homochiral units containing at least two or at least three polymerizable or crosslinkable functional groups are given below:

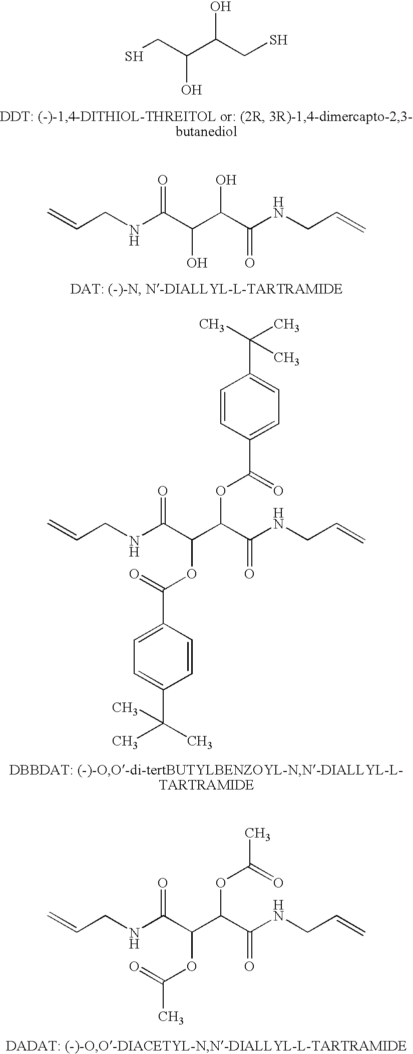

According to a second advantageous embodiment of the invention, the crosslinked optically active three-dimensional polymer network consists of at least one homochiral unit of at least one first selector and of at least one homochiral unit of at least one second selector of structure different from the first selector and of at least one homochiral unit of at least one third selector of structure different from the first and from the second selector, the homochiral unit(s) of the first selector containing one polymerizable functional group and the homochiral unit(s) of the third selector containing at least one polymerizable functionl groups, the homochiral units of the second selector containing at least two polymerizable functional groups, the homochiral units being chemically linked to one another.

Of course, the number of homochiral selectors of different structures is not limited to three, it may be much higher.

The homochiral unit(s) of the first selector contain(s) one and only one polymerizable functional group whereas the homochiral unit(s) of the third selector contain(s) either one or either two or even more polymerizable functional groups.

According to another advantageous embodiment of the polymer network in accordance with the invention, attached to at least some of the homochiral units of a selector chosen from the group comprising the first selector, the second selector and, optionally, the third selector, is a nonchiral crosslinking agent containing at least two polymerizable or crosslinkable functional groups.

The crosslinking agent containing at least two polymerizable or crosslinkable functional groups is chosen from the group comprising in particular ethanedithiol, trithiocyanuric acid, 1,6-hexanedithiol, 1,2,6-hexanetrioltrithioglycolate and 2,5-dimercapto-1,3,4-thiadiazole.

According to another advantageous embodiment, in the polymer network in accordance with the invention, the homochiral units of at least one of the selectors are β-cyclodextrin derivatives.

Thus, according to this particular embodiment, the polymer network may contain either units of a monofunctional derivative of β-cyclodextrin, i.e. derivative of β-cyclodextrin in which 1-OH goup has been replaced with a polymerizable functional group, and/or units of a bifunctional derivative of β-cyclodextrin, i.e. a derivative of β-cyclodextrin in which at least 2-OH groups have each been replaced with a polymerizable or crosslinkable functional group, and optionally units of a derivative of β-cyclodextrin in which more than 2-OH groups have each been replaced with a polymerizable or crosslinkable functional group.

The invention also relates to a method for preparing the crosslinked optically active polymer network.

Accordingly, the crosslinked optically active polymer network according to the first embodiment of the invention, are prepared using a method wherein:

a) at least one first selector consisting of one homochiral unit containing one polymerizable functional group, at least one second selector consisting of at least one homochiral unit containing at least two polymerizable or crosslinkable functional groups are selected;

b) optionally, at least one nonchiral crosslinking agent containing at least two polymerizable or crosslinkable functional groups is selected;

c) optionally, at least the homochiral unit of the first selector and/or at least some of the second selector are reacted with the nonchiral crosslinking agent;

d) either the homochiral unit of the first selector is copolymerized with the homochiral units of the second selector and;

e) or else at least some of the homochiral units containing at least two polymerizable or crosslinkable functional groups of the second selector are homopolymerized, and the homopolymerizates obtained are copolymerized with the homochiral unit of the first selector and optionally crosslinked with the remaining homochiral units containing at least two polymerizable or crosslinkable functional groups of the second selector.

Accordingly, the crosslinked optically active polymer network according to the second embodiment of the invention, are prepared using a method wherein:

a) at least one first selector consisting of at least one homochiral unit containing one polymerizable functional group, at least one second selector consisting of at least one homochiral unit containing at least two polymerizable or crosslinkable functional groups and at least one third selector consisting of at least one homochiral unit containing at least one polymerizable or crosslinkable functional group are selected;

b) optionally, at least one nonchiral crosslinking agent containing at least two polymerizable or crosslinkable functional groups is selected;

c) optionally, at least some of the homochiral units of the first selector and/or of the second selector and/or, of the third selector are reacted with the nonchiral crosslinking agent;

d) either the homochiral units of the first selector are copolymerized with the homochiral units of the second selector and with the homochiral units of the third selector;

e) or else at least some of the homochiral units containing one polymerizable or crosslinkable functional group of the first selector are homopolymerized, and the homopolymerizates obtained are crosslinked with the homochiral units containing at least two polymerizable or crosslinkable functional groups of the second selector and of the third selector, optionally in the presence of the remaining homochiral units of the first selector.

According to the particular embodiments including steps b) and c), in steps d) and e), use is made of at least some homochiral units of the first selector and/or of the second selector and/or, optionally, of the third selector to which the crosslinking agent is attached.

When it is desired to use a synthetic optically active polymer as one of the homochiral selectors, before carrying out the crosslinking operation with one or more other homochiral selectors, it is possible to use all the techniques described in the work by Eric Selegny entitled “Optically active polymers”, integrated into the series of works “Charged and reactive polymers”, volume 5, published in 1979 by D. Reidel Publishing Company, Dorrecht, Post Office Box 17, The Netherlands.

The invention also relates to an optically active support material, the optical activity properties of which are due to the fact that it consists in part of the polymer network described above.

The optically active support material in accordance with the invention consists of at least 0.1 to 100% of said optically active three-dimensional polymer network. The remainder up to 100% is generally in the form of silica gels, or of solid particles of mineral origin, such as silicon oxide, titanium oxide, aluminum oxide, clays, or of organic origin, such as polystyrenes, polyvinyl alcohols, etc.

The silica gels are the preferred supports when it is desired to use the final support material as CSP for enantioselective chromatography.

In accordance with the invention, the polymer network is either chemically linked to the mineral or organic support, or is physically deposited into the pores of the support, as described in the patents mentioned in the prior art. In the first case, the support undergoes prior chemical conversion making it possible to introduce functions capable of reacting and creating covalent bonds with the selectors of the polymer network.

The invention also relates to the use of an optically active support material containing the crosslinked three-dimensional polymer network described above, for removing from a mixture of at least two constituents, chosen from the group comprising organic, mineral or organomineral-molecules, at least most of one of these constituents. It is in fact an operation of purification by simply bringing the various constituents into contact with the support materials containing the crosslinked three-dimensional polymer network, which trap impurities, for example, or which, on the contrary, preferentially retain the desired constituent. The support materials may also be used as a stationary phase for separating said constituents by a chromatographic method.

The chromatographic methods use a simple column or a multicolumn system according to the “simulated mobile bed” technique.

The invention also relates to the use of an optically active support material containing the crosslinked three-dimensional polymer network described above, for removing from a mixture of at least two enantiomers, chosen from the group comprising chiral organic molecules or chiral organomineral molecules, at least part of one of these constituents, so as to enrich the mixture in one of the optically active homochiral molecules and to thus obtain one of the enantiomers enriched. The method used may be simply bringing said optically active support material into contact with the mixture of enantiomers, one of the enantiomers being preferentially adsorbed. The optical enrichment operation is carried out by filtration of the complex (optically active support material/adsorbed enantiomer). The complex is then destroyed by bringing it into contact with a liquid which is a solvent for said enantiomer and which has the property of eliminating the specific interaction of said enantiomer with the optically active support material. The desorbed enantiomer is either not used since it is of no value and, in this case, it is the first filtrate which is optically enriched in the desired enantiomer, or it is used as optically enriched enantiomer.

The invention also relates to the use of an optically active support material as an enantioselective stationary phase for separating optically active molecules by a chromatographic method. This technique is also advantageous as a method for producing optically or enantiomerically pure or enriched homochiral molecules.

The invention also relates to the use of the polymer network according to the invention, optionally in the presence of a transition metal, as catalyst for enantioselective synthesis. As examples of enantioselective synthesis, stereoselective reduction of carbonyl functions or reactions involving the formation of carbon-carbon bonds, as previously described inf FR2816948, can be cited.

### EXAMPLES

### Example 1

### (S)-glycidyl methacrylate-(2R,3R)-butanediol dimethacrylate copolymer further modified by N-benzylamine

5 g of (2R,3R)-butanediol (marketed product) are dissolved in 50 ml of anhydrous triethylamine and 10 ml of methacryloyl chloride are added in 3 hours between 0 and +5° C. The reaction medium is stirred during 5 hours at room temperature and then cooled again to 0, +5° C. 20 ml of water are added in 3 hours while maintaining the temperature lower than 20° C. (2R,3R)-butanediol dimethacrylate is extracted 3 times with 30 ml of methylene chloride. The chloromethylene solution is dried. The weight of the residue is 12.1 g, i.e. a yield of 96% (theoretical weight: 12.55 g).

The copolymerization of (S)-glycidyl methacrylate and (2R,3R)-butanediol dimethacrylate is carried out in teh presesnce of a free radical initiator according to the suspension polymerizaion meethod and the conditions of the synthesis are the one escribed in the French patent 2 816 948 (examples 1 à4). The obtained polymer

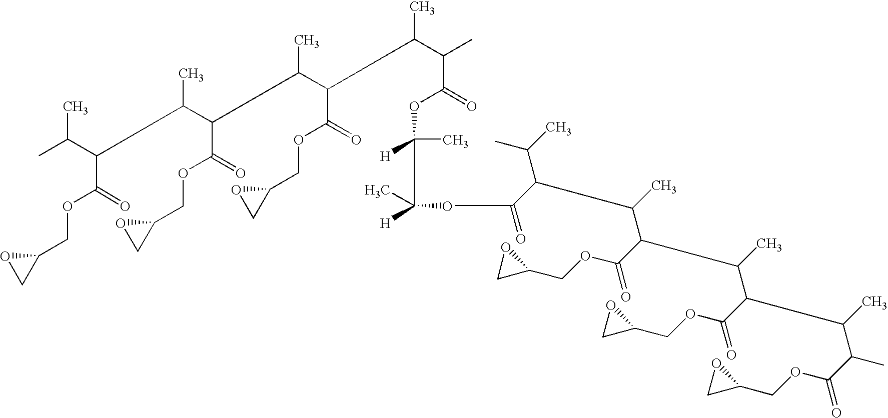

has the following chemical structure:

Elemental analysis: C: 59.7%; H: 7.4%; O: 32.8%. The functionality in epoxide functions is 2.1 meq/g.

The pur polymer pellets are separated depending on their size according to example 3 of the patent in question (use of sieves of 500, 300 et 106 μm).

The polymer is then modified by the action of benzylamine according to the procedure of example 4, using pellets with a mean diameter of 106 to 300 μm.

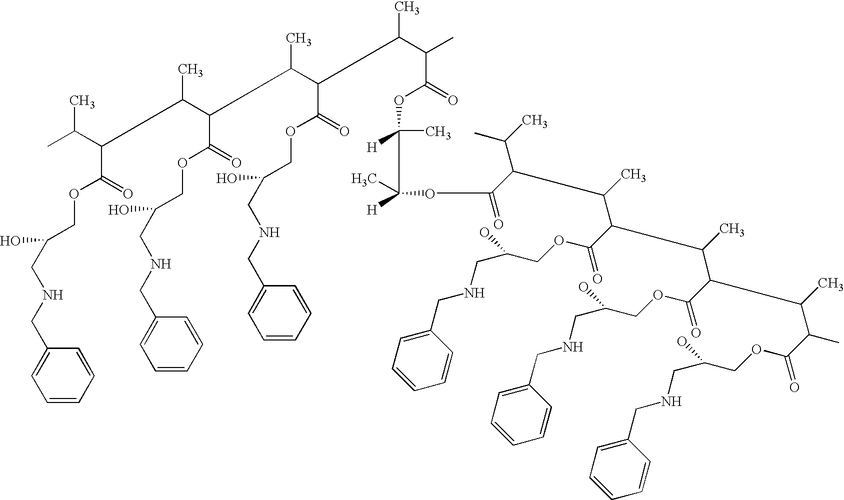

The polymer, the structure of which is represented above presents a functionalization rate of 1.12 mmol/g of polymer.

Elemental microanalyse: C: 59.7%; H: 7.2%; N: 1.50%.

A catalyst complex based on di-(paracymene)Ruthenium dichloride and above polymer is prepared in the conditions of example 5 of FR 281948.

Its use in the asymmetric reduction of acetophenone is performed according o example 6 of said patent.

The reduction reaction is as follows:

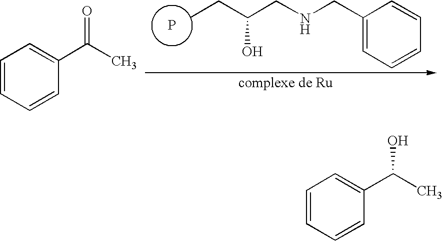

Acétophenone and the polymer comprising the Ruthenium complex are added in order to obtain a ratio acetophenone/mtal 20/1. 0.03 mol/L of potassium tertio-butylate in isopropanol are added with a ratio Ruthenium/tertio-butylate=1/5. The reaction mixture is stirred during 3 hours.

The enantiomeric excess of 1-phenylethanol obtained is measured by gas chromatographie on a chiral column SUPELCO beta-Dex-225 (30 m×25 mm). It is obtained with an enantiomeric excess of 75% and a conversion of 95%.

### Example 2

### Synthesis of a mono-6-O-(4-allyloxyphenylcarbamate)-hexakis-6-O-(3,5-dimethylphenylcarbamate)-diheptakis-2,3-O-(3,5-dimethylphénylcarbamate) and ditertiobutylbenzoyl diallyl tartramide copolymer

Mono-6-O-(4-allyloxyphenylcarbamate)-hexakis-6-O-(3,5-dimethylphenylcarbamate)-diheptakis-2,3-O-(3,5-dimethylphenylcarbamate) is copolymerized with ditertiobutylbenzoyl diallyl tartramide (configuration 2S, 3S), in the prsence of silica gel, after precipitation of the reactants into the silica gel pores, according to the following procedure: 0.25 g of mono-6-O-(4-allyloxyphenylcarbamate)-hexakis-6-O-(3,5-dimethylphenylcarbamate)-diheptakis-2,3-O-(3,5-dimethylphenylcarbamate) are dissolved in 10 ml of THF. 3 g of Kromasil silica, 5 μm (pore diameter 20 nm) are added and the obtained suspension is homogeneised. 0.15 g of ditertiobutylbenzoyl diallyl tartramide (configuration 2S, 3S) in solution in 5 ml of THF are added to the former suspension. 200 ml of heptane are dropped in 6 hours. The suspension is filtered and the insoluble is taken out in a wet state in 100 ml of heptane. 0.05 g of AIBN (azo-bis-isobutyronitrile, free radicals initiator) are added and the suspension is refluxing during 6 hours. 0.05 g of AIBN are again added and the suspension is refluxing during 6 hours. The mass is cooled and the suspension is filtered on sintered filter n°5. The insoluble is washed 3 times with 50 ml of boiling THF and 3 times with 50 ml of boiling methylene chloride. The insoluble is dried a 80° C. Dry weight=3.35 g.

Elemental Microanalysis: C % 15.19; H % 1.65; N % 1.16. 3 g are used to fill a HPLC column of 250 mm (length)×4.6 mm (internal diamter). The column is conditioned in pure chloroforme. 1 μg f Indapamide is injected in the column (20 μl of a chloroform solution) an dis eluted in pure chloroforme pur with a flow rate of 1 ml/mn. The detection wavelength is 254 nm and the scale of the optical density is 0.2. The dead time measured with sodium azide de sodium is of 3′. Retention factors are k′1=11,′ and k′2=14.7. Enantiosélectivity rate α=k′2/k′1 is of 1.29.

1. A crosslinked optically active three-dimensional polymer network consisting of one homochiral unit of at least one first selector and of at least one homochiral unit of at least one second selector of a structure different from the first selector, the homochiral unit of the first selector containing one polymerizable functional group and the homochiral unit(s) of the second selector containing at least two polymerizable or crosslinkable functional groups,
the homochiral units being chemically linked to one another,
with the exclusion of the crosslinked three-dimensional polymer networks obtained by polymerization of (S)-glycidylmethacrylate and simultaneous cross-linking with (S,S)-2,3-butanediol dimethacylate or by polymerization of 3-([2-(S)-hyroxy]-N-benzylamino)propylmethacrylate and simultaneous cross-linking with (S,S)-2,3-butanediol methacrylate.

2. A crosslinked optically active three-dimensional polymer network consisting of at least one homochiral unit of at least one first selector and of at least one homochiral unit of at least one second selector of structure different from the first selector and of at least one homochiral unit of at least one third selector of structure different from the first and from the second selector, the homochiral unit(s) of the first selector containing one polymerizable functional group, the homochiral unit(s) of the third selector containing at least one polymerizable functional group and the homochiral unit(s) of the second selector containing at least two polymerizable or crosslinkable functional groups, the homochiral units being chemically linked to one another.

3. The polymer network according to claim 1 wherein a nonchiral crosslinking agent containing at least two polymerizable or crosslinkable functional groups is attached to at least some of the homochiral units of a selector chosen from the group comprising the first selector, the second selector and, optionally, the third selector.

4. The polymer network according to claim 1, wherein the homochiral unit of the first selector and, optionally, the homochiral units of the third selector are chosen from the group comprising in particular mono-6-O-(4-allyloxyphenylcarbamate)-hexakis-6-O-(3,5-dimethylphenylcarbamate)-di-heptakis-2,3-O-(3,5-dimethylphenylcarbamate)-β-cyclodextrin, 2-propynyl-tetra-O-acetyl-β-glucopyranoside, allyl-α-D-galactopyranoside, 1-O-allyl-2-deoxy-4,6-O-isopropylidene-2-(trifluoroacetamido)-α-D-galacto pyranoside, 7-allyl-7,8-dihydro-8-oxoguanosine, (R)-(+)-α-acryloxy-β,β-dimethylbutyrolactone, acrylamido-(L)-alanine ethyl ester, (2S,5R)-(+)-5-vinyl-2-quinuclidinemethanol, (2R,5R)-( )-5-vinyl-2-quinuclidinemethanol, quinine and quinidine.

5. The polymer network according to claim 1, wherein the homochiral units of the second selector and, optionally, of the third selector are chosen from the group comprising in particular (R,R)-dithiothreitol (DTT), tartaric acid or derivatives thereof, such as N,N′-diallyltartramide (DAT), di-tert-butylbenzoyldiallyltartramide (DBBDAT), diacetyldiallyltartramide (DADAT), bi-derivatives of cyclodextrin, in particular β-cyclodextrin, such as bis-6A,6D-O-(4-allyloxyphenylcarbamate)pentakis-6-O-(3,5-dimethylphenylcarbamate)-di-heptakis-2,3-(3,5-dimethylphenylcarbamate)-β-cyclodextrin.

6. The polymer network according to claim 3, wherein the nonchiral crosslinking agent containing at least two polymerizable or crosslinkable functional groups is chosen from the group comprising in particular ethanedithiol, trithiocyanuric acid, 1,6-hexanedithiol, 1,2,6-hexanetrioltrithioglycolate and 2,5-dimercapto-1,3,4-thiadiazole.

7. The polymer network according to claim 1, in which the homochiral units of at least one of the selectors are β-cyclodextrin derivatives.

8. The polymer network according to claim 2 wherein a nonchiral crosslinking agent containing at least two polymerizable or crosslinkable functional groups is attached to at least some of the homochiral units of a selector chosen from the group comprising the first selector, the second selector and, optionally, the third selector.

9. The polymer network according to claim 2, wherein the homochiral units of the first selector and, optionally, of the third selector are chosen from the group comprising in particular mono-6-O-(4-allyloxyphenylcarbamate)-hexakis-6-O-(3,5-dimethylphenylcarbamate)-di-heptakis-2,3-O-(3,5-dimethylphenylcarbamate)-β-cyclodextrin, 2-propynyl-tetra-O-acetyl-β-glucopyranoside, allyl-α-D-galactopyranoside, 1-O-allyl-2-deoxy-4,6-O-isopropylidene-2-(trifluoroacetamido)-α-D-galacto pyranoside, 7-allyl-7,8-dihydro-8-oxoguanosine, (R)-(+)-α-acryloxy-β,β-dimethylbutyrolactone, acrylamido-(L)-alanine ethyl ester, (2S,5R)-(+)-5-vinyl-2-quinuclidinemethanol, (2R,5R)-( )-5-vinyl-2-quinuclidinemethanol, quinine and quinidine.

10. The polymer network according to claim 2, wherein the homochiral units of the second selector and, optionally, of the third selector are chosen from the group comprising in particular (R,R)-dithiothreitol (DTT), tartaric acid or derivatives thereof, such as N,N′-diallyltartramide (DAT), di-tert-butylbenzoyldiallyltartramide (DBBDAT), diacetyldiallyltartramide (DADAT), bi-derivatives of cyclodextrin, in particular β-cyclodextrin, such as bis-6A,6D-O-(4-allyloxyphenylcarbamate)pentakis-6-O-(3,5-dimethylphenylcarbamate)-di-heptakis-2,3-(3,5-dimethylphenylcarbamate)-β-cyclodextrin.

11. The polymer network according to claim 8, wherein the nonchiral crosslinking agent containing at least two polymerizable or crosslinkable functional groups is chosen from the group comprising in particular ethanedithiol, trithiocyanuric acid, 1,6-hexanedithiol, 1,2,6-hexanetrioltrithioglycolate and 2,5-dimercapto-1,3,4-thiadiazole.

12. The polymer network according to claim 2, in which the homochiral units of at least one of the selectors are β-cyclodextrin derivatives.

13. A method for preparing a polymer network according to claim 1, wherein:
a) at least one first selector consisting of one homochiral unit containing one polymerizable functional group, at least one second selector consisting of at least one homochiral unit containing at least two polymerizable or crosslinkable functional groups are selected;
b) either the homochiral unit of the first selector is copolymerized with the homochiral units of the second selector and;
c) or else at least some of the homochiral units containing at least two polymerizable or crosslinkable functional groups of the second selector are homopolymerized, and the homopolymerizates obtained are copolymerized with the homochiral unit of the first selector and optionally crosslinked with the remaining homochiral units containing at least two polymerizable or crosslinkable functional groups of the second selector.

14. A method for preparing a polymer network according to claim 3, wherein:
a) at least one first selector consisting of one homochiral unit containing one polymerizable functional group, at least one second selector consisting of at least one homochiral unit containing at least two polymerizable or crosslinkable functional groups are selected;
b) at least one nonchiral crosslinking agent containing at least two polymerizable or crosslinkable functional groups is selected;
c) at least the homochiral unit of the first selector or at least some of the second selector are reacted with the nonchiral crosslinking agent;
d) either the homochiral unit of the first selector is copolymerized with the homochiral units of the second selector and;
e) or else at least some of the homochiral units containing at least two polymerizable or crosslinkable functional groups of the second selector are homopolymerized, and the homopolymerizates obtained are copolymerized with the homochiral unit of the first selector and optionally crosslinked with the remaining homochiral units containing at least two polymerizable or crosslinkable functional groups of the second selector.

15. A method for preparing a polymer network according to claim 2, wherein:
a) at least one first selector consisting of at least one homochiral unit containing one polymerizable functional group, at least one second selector consisting of at least one homochiral unit containing at least two polymerizable or crosslinkable functional groups and at least one third selector consisting of at least one homochiral unit containing at least one polymerizable or crosslinkable functional group are selected;
b) either the homochiral units of the first selector are copolymerized with the homochiral units of the second selector and with the homochiral units of the third selector;
c) or else at least some of the homochiral units containing one polymerizable or crosslinkable functional group of the first selector are homopolymerized, and the homopolymerizates obtained are crosslinked with the homochiral units containing at least two polymerizable or crosslinkable functional groups of the second selector and of the third selector, optionally in the presence of the remaining homochiral units of the first selector.

16. A method for preparing a polymer network according to claim 8, wherein:
a) at least one first selector consisting of at least one homochiral unit containing one polymerizable functional group, at least one second selector consisting of at least one homochiral unit containing at least two polymerizable or crosslinkable functional groups and at least one third selector consisting of at least one homochiral unit containing at least one polymerizable or crosslinkable functional group are selected;
b) at least one nonchiral crosslinking agent containing at least two polymerizable or crosslinkable functional groups is selected;
c) at least some of the homochiral units of the first selector or of the second selector or of the third selector are reacted with the nonchiral crosslinking agent;
d) either the homochiral units of the first selector are copolymerized with the homochiral units of the second selector and with the homochiral units of the third selector;
e) or else at least some of the homochiral units containing one polymerizable or crosslinkable functional group of the first selector are homopolymerized, and the homopolymerizates obtained are crosslinked with the homochiral units containing at least two polymerizable or crosslinkable functional groups of the second selector and of the third selector, optionally in the presence of the remaining homochiral units of the first selector.

17. An optically active support material containing a polymer network according to claim 1, and an inert, mineral or organic support, said support preferably being in the form of solid particles.

18. The support material according to claim 9, consisting of at least 0.1% by weight of the polymer network.

19. The support material according to claim 9 wherein the polymer network is chemically linked to the support or is deposited onto the support.

20. An optically active support material containing a polymer network according to claim 2, and an inert, mineral or organic support, said support preferably being in the form of solid particles.

21. The support material according to claim 20, consisting of at least 0.1% by weight of the polymer network.

22. The support material according to claim 20, wherein the polymer network is chemically linked to the support or is deposited onto the support.

23. Method for the purification of a mixture of at least two constituents, chosen from the group comprising organic, mineral or organomineral molecules, wherein the various constituents are brought into contact with a polymer network according to claim 1 and at least part of one of these constituents is removed.

24. Method for removing from a mixture of at least two enantiomers, chosen from the group comprising chiral organic molecules or chiral organomineral molecules, at least part of one of these constituents, wherein the mixture is brought into contact with the polymer network according to claim 1 in order to form a polymer network/absorbed enantiomer complex, said complex is then filtered and destroyed with a solvent for said enantiomer, so as to enrich the mixture in one of the optically active homochiral molecules and to thus obtain one of the enantiomers enriched.

25. Method for separating optically active molecules by chromatographic method, wherein the stationary phase is the polymer network according to claim 1.

26. Method for the purification of a mixture of at least two constituents, chosen from the group comprising organic, mineral or organomineral molecules, wherein the various constituents are brought into contact with a polymer network according to claim 2 and at least part of one of these constituents is removed.

27. Method for removing from a mixture of at least two enantiomers, chosen from the group comprising chiral organic molecules or chiral organomineral molecules, at least part of one of these constituents, wherein the mixture is brought into contact with the polymer network according to claim 2 in order to form a polymer network/absorbed enantiomer complex, said complex is then filtered and destroyed with a solvent for said enantiomer, so as to enrich the mixture in one of the optically active homochiral molecules and to thus obtain one of the enantiomers enriched.

28. Method for separating optically active molecules by chromatographic method, wherein the stationary phase is the polymer network according to claim 2.

29. Method of asymmetric synthesis wherein the synthesis is performed with a polymer network according to claim 1, optionally in the presence of a transition metal.

30. Method of asymmetric synthesis wherein the synthesis is performed with a polymer network according to claim 2, optionally in the presence of a transition metal.
